(19) United States
(12) Patent Application Publication
(10) Pub. No.: US 2026/0053443 A1
(43) Pub. Date: Feb. 26, 2026

(54) Flexible Hybrid Circuits, Along with Systems of Embedded Processing and Wireless Communications

(71) Applicant: California State University San Marcos Corporation, San Marcos, CA (US)

(72) Inventors: Reza Kamali-Sarvestani, San Diego, CA (US); Suzanne Moineau, San Marcos, CA (US)

(73) Assignee: California State University San Marcos Corporation, San Marcos, CA (US)

(21) Appl. No.: 19/309,189

(22) Filed: Aug. 25, 2025

Related U.S. Application Data
(60) Provisional application No. 63/686,801, filed on Aug. 25, 2024.

(51) Int. Cl. A61B 5/00

(52) U.S. Cl. CPC A61B 5/682; A61B 5/0022; A61B 2562/164

(57) ABSTRACT

Contemplated embodiments include a biomedical sensor system, comprising: a piezoresistive agent, and a flexible substrate, wherein the flexible substrate and the piezoresistive agent are coupled together to form the biomedical sensor system, and wherein the sensor system is removably located in or on the ceiling of a patient's mouth. Contemplated methods of determining a level of dysphagia in a patient include: providing a biomedical sensor system, wherein the system comprises: a piezoresistive agent, and a flexible substrate comprising a flexible hybrid circuit structure, wherein the flexible substrate and the piezoresistive agent are coupled together to form the biomedical sensor system; providing a patient having a mouth, a tongue, and a mouth ceiling, removably locating the sensor system in or on the ceiling of the patient's mouth.

This United States Utility patent application claims priority to U.S. Provisional Patent Application Ser. No. 63/686,801, which was filed on Aug. 25, 2024, which is commonly owned, and which is incorporated herein in its entirety by reference.

### FIELD OF THE SUBJECT MATTER

The field of the subject matter is flexible hybrid circuits, along with systems of embedded processing and wireless communications.

### BACKGROUND

Dysphagia, identified as a swallowing disorder, affects roughly one in every twenty-five individuals in the U.S., leading to significant health complications. The American Speech-Language-Hearing Association (ASHA) reports that about one-third of those suffering from dysphagia are at risk of developing pneumonia, a complication that results in the death of 60,000 people annually. A comprehensive review of adult dysphagia is recently published and shows the significance of required measurement in this field [1].

The laryngeal elevation during swallowing plays a crucial role in safeguarding the trachea and facilitating the opening of the upper esophageal sphincter. Our research aims to closely examine this elevation by developing a wearable oral device targeted at individuals suffering from aspiration pneumonia. This condition, which affects the lungs or airways, is predominantly observed in the elderly or those with compromised or immature immune systems.

Originating from dysphagia, or swallowing dysfunction, aspiration pneumonia occurs when substances like food, water, saliva, or vomit are accidentally breathed into the lungs instead of being directed through the esophagus to the stomach. The consequences of aspiration pneumonia include neurological impairments, respiratory issues, head and neck injuries, and cancer, among others, potentially leading to malnutrition and a significant decline in the quality of life over time.

The ultimate objective for our device is to achieve flexibility, durability, oral safety, compactness, and user comfort. It is designed to detect various oral parameters and alterations to accurately track the swallowing mechanism. This innovative tool aims to facilitate a non-invasive approach, ensuring patient comfort during treatment without the necessity for exposure to X-ray or video fluoroscopy imaging [2]. Currently, such a device is unprecedented in the medical industry.

Previous studies conducted by other researchers in this field have focused on measuring various oral parameters, including the anterior and posterior regions of the hard palate. They utilized triaxial force sensors equipped with piezoresistive hinges and silicone rubber (PDMS) for this purpose. Their findings indicated that the activity in the anterior part of the tongue was more significant than in the posterior part. This discovery sheds light on the optimal placement of sensors within the mouth and suggests that employing multiple sensors could effectively identify the areas of the tongue exerting greater force [3]. Images accompanying their research illustrate the dimensions, shape, and materials of the design, serving as a source of inspiration for further enhancements.

The first major category of sensors is using resistive sensors for oral measurement [3, 4]. Other sensory system research for swallowing are devices with sheets placed on the exterior part of a patient's neck [2]. In this study, we report the application of Single Wall Carbon Nanotubes (SW-CNT) as the piezoresistive agent and Kapton as a flexible substrate to make a comfortable sensor for patients to put in the ceiling of their mouth.

### SUMMARY OF THE SUBJECT MATTER

Contemplated embodiments include a biomedical sensor system, comprising: a piezoresistive agent, and a flexible substrate, wherein the flexible substrate and the piezoresistive agent are coupled together to form the biomedical sensor system, and wherein the sensor system is removably located in or on the ceiling of a patient's mouth. Contemplated embodiments also include a biomedical sensor system that includes: a piezoresistive agent, a flexible substrate, and a patient having a mouth, a tongue, and a mouth ceiling, wherein the flexible substrate and the piezoresistive agent are coupled together and/or integrated together to form the biomedical sensor system, and wherein the sensor system is removably located in or on the ceiling of the patient's mouth.

Contemplated methods of determining a level of dysphagia in a patient include: providing a biomedical sensor system, wherein the system comprises: a piezoresistive agent, and a flexible substrate comprising a flexible hybrid circuit structure, wherein the flexible substrate and the piezoresistive agent are coupled together to form the biomedical sensor system; providing a patient having a mouth, a tongue, and a mouth ceiling, removably locating the sensor system in or on the ceiling of the patient's mouth.

Contemplated embodiments may also comprise at least one additional active component. These at least one additional active components may comprise at least one of the following alone or in combination: batteries, resistors, carbon nanotubes, material adhesives, bioadhesives, wireless communication technology and components, microcontrollers, and related components.

### BRIEF DESCRIPTION OF THE FIGURES

FIG. 1 shows a biomedical sensor system, comprising: a piezoresistive agent, and a flexible substrate, wherein the sensor system can be removably located and/or removably placed and/or removably secured in/on the ceiling/roof of a patient's mouth.

FIG. 2 shows a photo of a hybrid flexible board designed and tested, as part of this work.

FIG. 3 shows a contemplated biomedical sensor system removably located in the ceiling of a patient's mouth.

FIG. 4 shows the trend of conductivity changes in response to applied pressure based on equation No. 1, emphasizing the potential of CNTs in pressure-sensing applications due to their significant piezoresistive response.

FIG. 5 shows a mouth set model. When we measured finger digit sensors covered each one by the 20-microliter SW-CNT, we observed resistance values ranging from 5 to 65 Ohms.

FIG. 6 shows the pressure applied at a separate time on each sensor, which can be used as a measurement of the functionality of sensors.

### DETAILED DESCRIPTION

Contemplated embodiments include a wireless sensory system using hybrid flexible electronics with embedded processing and wireless communication for a conformable sensing platform that integrates multiple flexible sensors with localized data processing and wireless connectivity to efficiently monitor and transmit environmental or physiological information in real time.

Contemplated embodiments include a biomedical sensor system, comprising: a piezoresistive agent, and a flexible substrate, wherein the flexible substrate and the piezoresistive agent are coupled together to form the biomedical sensor system, and wherein the sensor system is removably located in or on the ceiling of a patient's mouth.

Contemplated embodiments also include a biomedical sensor system that includes: a piezoresistive agent, a flexible substrate, and a patient having a mouth, a tongue, and a mouth ceiling, wherein the flexible substrate and the piezoresistive agent are coupled together and/or integrated together to form the biomedical sensor system, and wherein the sensor system is removably located in or on the ceiling of the patient's mouth.

Contemplated methods of determining a level of dysphagia in a patient include: providing a biomedical sensor system, wherein the system comprises: a piezoresistive agent, and a flexible substrate comprising a flexible hybrid circuit structure, wherein the flexible substrate and the piezoresistive agent are coupled together to form the biomedical sensor system; providing a patient having a mouth, a tongue, and a mouth ceiling, removably locating the sensor system in or on the ceiling of the patient's mouth.

Contemplated embodiments may also comprise at least one additional active component. These at least one additional active components may comprise at least one of the following alone or in combination: batteries, resistors, carbon nanotubes, material adhesives, bioadhesives, wireless communication technology and components, microcontrollers, and related components.

Contemplated sensors are located right above a microcontroller board with a Bluetooth connection to send the measurement date out to the computer or a new cellphone. This sensory system benefits from a hybrid flexible microcontroller board to bend it inside the mouth and make it easier for patients to wear the sensory system.

A microcontroller is being used as the main data acquisition processor. While in this disclosure we used a four-sensor structure, we made 8 input ports to make the capability of capturing the data of up to 8 sensors. The board has small components to help maintain the flexibility of the substrate and reduce the thickness of the circuit. The board communicates using Bluetooth with the outside of the mouth. There is no need for the connecting long wires to come out of the mouth. This is important to give the patient a more comfortable swallowing procedure. Flexible substrates have revolutionized the electronics industry by offering unprecedented versatility and durability in device manufacturing. The inherent flexibility of our substrate enables the creation of bendable, foldable, and even rollable electronics, opening new possibilities for wearable technology or a medical device for an oral sensory system application.

One of the primary benefits of contemplated flexible substrates is their ability to significantly reduce the weight and thickness of electronic devices compared to traditional rigid circuit boards, which can lead to a reduction in material usage by up to 60%. Hence, changes in resistance due to applied pressure directly affect conductivity. In piezoresistive materials, as resistance increases with pressure (or strain), conductivity decreases, and vice versa. device weight and improving portability and comfort for wearable technology compared to the same microcontroller board that we made without a flexible board.

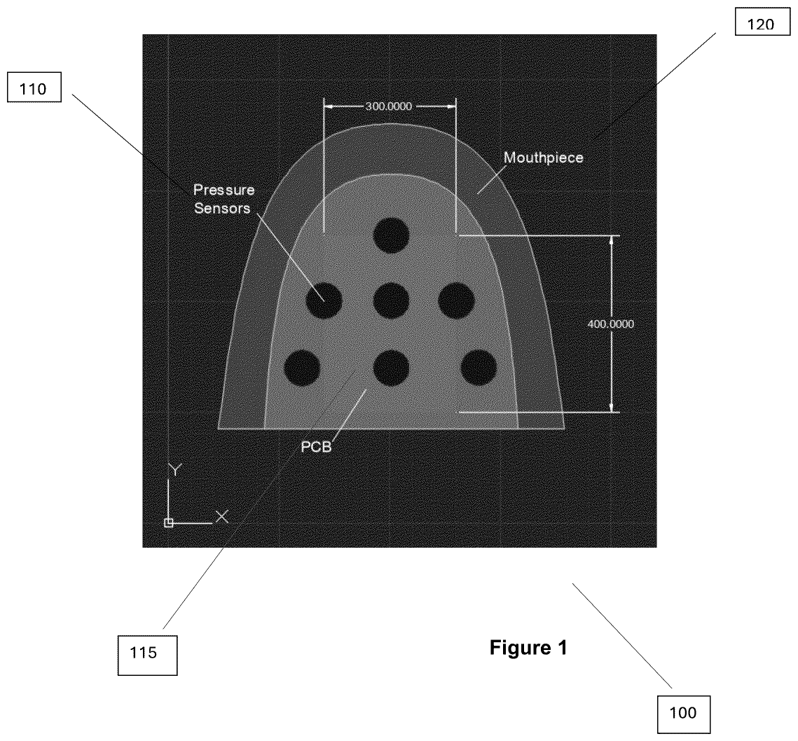

FIG. 1 shows a biomedical sensor system 100, comprising: a piezoresistive agent 110, a printed circuit board 115, and a flexible substrate 120, wherein the sensor system can be removably located and/or removably placed and/or removably secured in/on the ceiling/roof of a patient's mouth (not shown). Contemplated embodiments include a biomedical sensor system, comprising: a piezoresistive agent, and a flexible substrate, wherein the flexible substrate and the piezoresistive agent are coupled together and/or integrated together to form the biomedical sensor system, and wherein the sensor system is removably located in or on the ceiling of a patient's mouth.

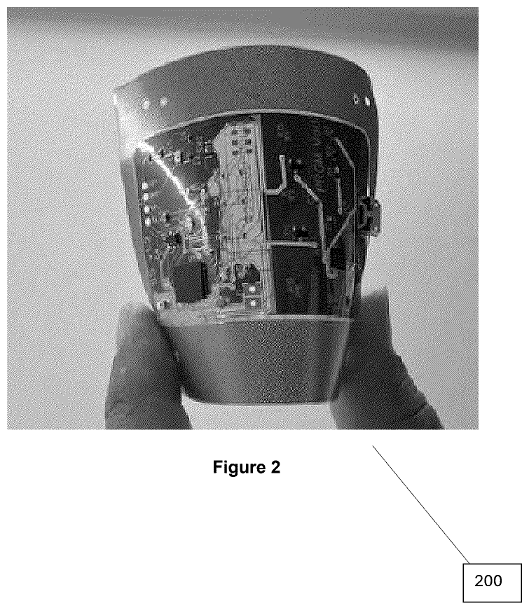

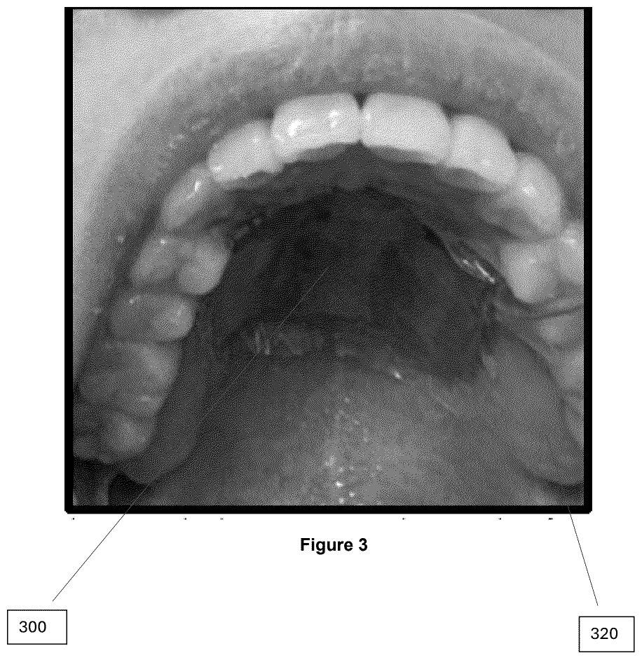

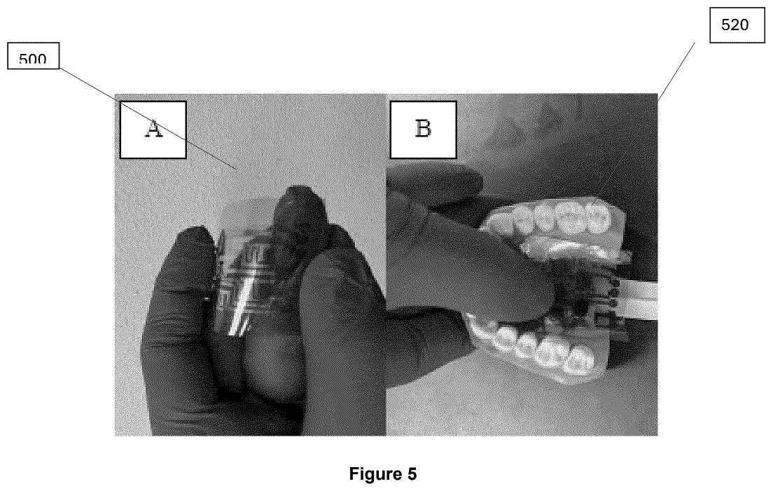

In contemplated embodiments, the piezoresistive agent is layered onto, coupled with, integrated with, or otherwise connected to the flexible substrate. In additional contemplated embodiments, biomedical sensor systems include at least one additional active component. Contemplated additional active components include at least one of the following alone or in combination: batteries, resistors (shown in FIGS. 2 and 5), carbon nanotubes (shown in FIGS. 2 and 5), material adhesives, bioadhesives (for use coupling the sensor device to the patient's mouth and shown in FIGS. 3 and 5), wireless communication technology and components (shown in FIG. 5), microcontrollers (shown in FIG. 5), and related components.

In some embodiments, the piezoresistive agent comprises a piezoresistive sensor. In additional embodiments, the piezoresistive sensor comprises at least one carbon nanotube. In other embodiments, the piezoresistive sensor comprises multiple carbon nanotubes. In contemplated embodiments, the piezoresistive sensor is activated or triggered electronically by mechanical deformation of at least one of the at least one carbon nanotube. In these embodiments, the mechanical deformation is caused by pressure on the ceiling of a patient's mouth by a tongue of the patient.

In some embodiments, the flexible substrate comprises a wireless embedded microcontroller system and a communications system. In these embodiments, the communications system comprises Bluetooth communications protocol.

As contemplated herein, the term “piezoresistive” refers to “a change in the electrical resistivity of a semiconductor or metal when mechanical strain is applied”. In contrast to the piezoelectric effect, the piezoresistive effect causes a change only in electrical resistance, not in electric potential. As used herein, the phrase “piezoresistive agent” refers to a semiconductor or metal that exhibits a change in the electrical resistivity when mechanical strain is applied to the semiconductor or metal.

FIG. 2 shows a photo of a hybrid flexible board 200 designed and tested, as part of this work. FIG. 3 shows a contemplated biomedical sensor system 300 removably located in/on the ceiling of a patient's mouth 320. The manufacturing of flexible electronics involves several cutting-edge techniques, among which the most prominent are roll-to-roll (R2R) processing and inkjet printing. R2R processing is similar to printing newspapers on a massive scale, enabling the cheaper mass production of sensors or devices.

The theoretical description of the effect of pressure on carbon nanotube (CNT) resistive or piezoresistive sensors involves understanding how mechanical deformation (due to pressure) alters the electrical resistance (or conductivity) of the CNTs. CNTs are known for their exceptional mechanical and electrical properties, making them excellent candidates for piezoresistive sensor applications. When pressure is applied, it deforms the CNTs, affecting their electronic band structure and hence their electrical resistance.

The piezoresistive effect in CNTs can be quantitatively described by the gauge factor (GF), which relates to the relative change in resistance (ΔR/R0) to the applied mechanical strain (ε) [5]:

$$G F = ( Δ R / R 0 ) / ε \qquad ( 1 )$$

where: GF is the gauge factor, ΔR=R−R0 is the change in resistance, R0 is the initial resistance, R is the resistance under strain, and & is the mechanical strain induced by the applied pressure. For CNT-based sensors, the strain (8) can be related to applied pressure (P) through the material's mechanical properties, such as Young's modulus (E) and the Poisson ratio (v), assuming a linear elastic behavior for simplicity [6].

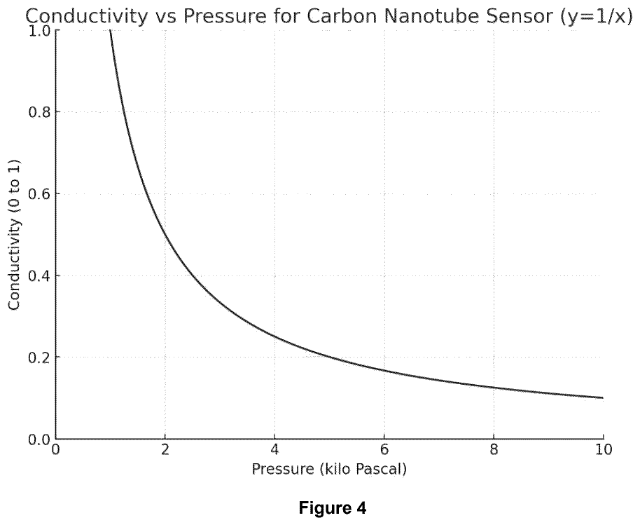

FIG. 4 shows the trend of conductivity changes in response to applied pressure based on equation No. 1, emphasizing the potential of CNTs in pressure-sensing applications due to their significant piezoresistive response. Measuring tongue pressure in the mouth for swallowing disorder assessment involves several basic elements crucial for accurately evaluating and diagnosing conditions such as dysphagia. At the core of this process is the pressure sensor that would be fixed at the ceiling of the mouth. We used four sensors for the measurement. These sensors were already tested to have enough sensitivity to capture subtle variations in pressure that occur during the complex act of swallowing.

The data collected by the sensors is then wirelessly transmitted using Bluetooth to the cellphone. In our design, we have a battery outside of the mouth and a thin wire takes electric power to the circuit inside the mouth. The integration of such sensors into a clinical device helped to have a noninvasive measurement of tongue pressure.

FIG. 5 shows a mouth set model/contemplated embodiment 500 that can then be removably inserted into or onto the roof of a patient's mouth 520. When we measured finger digit sensors covered each one by the 20-microliter SW-CNT, we observed resistance values ranging from 5 to 65 Ohms.

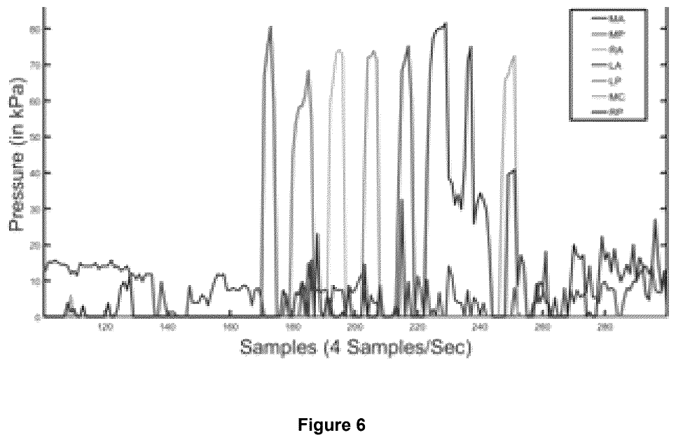

FIG. 6 shows the pressure applied at a separate time on each sensor, which can be used as a measurement of the functionality of sensors. We applied force by placing standard weights on the sensors. Additionally, we validated these measurements by placing the sensors on a digital scale and directly measuring the force applied to each sensor. The microcontroller board is being powered by a 3.7 V battery external to the board. Battery is staying out of mouth and it is connected to the board using a wire.

Our measurement of four sensors shows that the sensors are very sensitive to forces from 0 to 25 Newton. Then the sensitivity will drop and the sensor will not be efficient for forces above 35 Newton because the substrate was not moving flexibly vertically. If more force is going to be measured a larger sensor footprint, a thick deposition of SWCNT, or a vertically flexible sensor will be required. Our current structure is good enough for regular tongue forces.

Measuring tongue pressure in the mouth for swallowing disorder assessment requires a flexible sensory system. We designed and fabricated a flexible four-sensor structure and a hybrid flexible wireless data acquisition board to transfer the data. The sensory system shows the possibility of making flexible wearable biomedical systems for noninvasive measurements for internal body measurements.

### Examples

This project involved designing and creating a prototype device for use in diagnosing a condition known as Dysphagia, which is a condition that affects a subject's swallowing or drinking abilities. The device is meant to be removably placed on the subject's upper palate (roof or ceiling of the mouth) and is designed to take pressure readings of tongue movements, so that any irregularities can be observed on an application, through wireless communication, such as Bluetooth.

Previous designs of contemplated biomedical sensor devices involved making the device more comfortable for the test subject, incorporating wireless communication, and adding more sensors. However, the sensors in the previous design were stiff and large, they also were wrapped in Kapton tape for waterproofing, all leading to the mouth module being very uncomfortable for the user. The mouthguard design also would interfere with the subject's mouth if they were to chew or drink, defeating the purpose of the device's testing purposes. The device also was hard wired into the computer via USB and had large uncomfortable wires leading to each of the sensors. To be easier to transport and set up, the device should ideally be capable of wireless communication via Bluetooth, as well as function with a battery. Contemplated sensors need to be small enough to provide a good resolution of the mouth's movement and sensitive to detect small pressures. Sensors in the market have not been found to meet those needs, so contemplated biomedical sensor systems had to be created.

Using online software, such as a computer-aided printed circuit board design software package, new boards were designed and ordered. Simultaneously, research into new possible sensor types was conducted. The application to display data was developed for Windows and was proven to be functional with the previous prototype. Contemplated designs incorporate and/or comprise pads on its surface (the flexible substrate) so that materials can be applied, creating on-board sensors. Research was performed to find appropriate materials to use that have the proper resistance values for operation, and a successful combination was found, leading to exceptional results.

Design objectives included:
Design a Microcontroller PCB with the requested specifications:
Board must be both small & flexible, limiting it to a two-layer PCB layout using the new millimeter size microcontroller.
Device should be able to communicate wirelessly, so it must have an on-board antenna for Wi-Fi or Bluetooth capabilities.
Connections must be provided to support at least seven sensor inputs.
Power must be supplied by a battery, and should have a small footprint, as well.
Research and implement new sensors in design:
   Sensors should have a small profile and should not discomfort the test subject.
   Sensors should be capable of taking in pressure data and accurately convey that information to the device.
   Research into creation of new sensors, in-house, may have to be conducted.
Complete development on an application to display data:
   The application must be able to communicate with the microcontroller wirelessly, to receive the data from the microcontroller.

The previous prototype created for this project was a rigid PCB, running a ESP-32 microcontroller, that connected to a PC via USB connection and had long wires attached to a retainer, holding several FSRs. Due to complaints about the impracticality of this design, the next iteration would require a full return to the drawing board.

A redesigned PCB was built around the microcontroller, which has Bluetooth functionality, and boasts a smaller footprint (6 mm×6 mm). This iteration was designated the FSS (Flexible Sensory System) v1.1. To add flexibility to the PCB, the number of layers were limited to two.

The PCB drafting software was used to create the new schematic and board design. Some mini 1.25-1.27 pitch header pins were added to the board to interface with the sensors and to provide a means to program the microcontroller. An antenna was also created for the 2.4 GHz Bluetooth communication capabilities. To allow for tracks to fit and have plenty of room to be routed, the track width was kept at 0.160 mm. Contemplated traces were both routed automatically and routed manually, which was quite time consuming, but was completed successfully. Ideally, contemplated traces would be routed automatically, but it should be understood that contemplated traces may be routed manually or routed using a combination of both automatic and manual routing. A contemplated design came out to be 50 mm×50 mm. PCB Design was completed and five boards were submitted for fabrication.

For the power supply of the device, a Molex flexible 3 V battery was considered, because of its small and flat dimensions, as well as its range of flexibility. Depending on how all the device components will be packaged in the end that will determine if we proceed with this battery or use a coin cell battery, as in the previous prototype.

The PCBs were received and appeared exactly as the design specified. In the engineering lab, the board was powered by a power supply with 3.3 VDC at the input ports to verify that the correct voltages were observed in the circuit. No issues were observed with the route tracing, and components seemed to stay fastened after flexing the board significantly, which was very promising for the reliability of the design.

With respect to a microcontroller, the previous design incorporated an ESP32 microcontroller, which was easily compatible with Arduino IDE and programmer. The updated design used the microcontroller, which required a more involved programming process. A development kit was used in conjunction with the semiconductor interfacing software.

Goals for additional contemplated embodiments were primarily centered around downsizing even further to 30 mm×40 mm while retaining the same capabilities. To keep the design of the PCB flat, the header pins were removed from the design, leaving some copper pads to attach the PCB sensor leads to, instead. The sole header pins that will be included will be attached to a small strip coming off the PCB, which will serve as the programming and debugging interface. The strip will then be able to be snipped off, after successful programming, so that the PCBs footprint remains minimal.

A contemplated MCU powered device was then fabricated. The system was functional and worked well with the sensors, however it had some initial power issues that needed to be addressed. It was discovered that the device requires about 250 mA for a very short amount of time, to successfully power on. This makes most small coin-cell batteries not applicable for this, and so a bigger battery was required. A larger Li-Ion 3.7v battery proved to be plenty and a compromise was made: instead of having the battery fit in the mouth with the design, it would be connected with a very small wire that would hang outside the mouth, with the subject holding the battery.

The device was detected by a phone Bluetooth and transmitted values successfully.

For an additional contemplated design, it was proposed that the FSS have copper fingers printed on its surface. This was in order to experiment with creating sensors on the PCB by dropping carbon nano tubing or some other material in the exposed substrate between these copper fingers, thus eliminating the need for a separate structure.

FSS v2.1 operated successfully with the Windows desktop application, upon being programmed with the Bluetooth conventional code. Further research was conducted with sensor materials and a suitable combination of carbon nanotubes and polyethylene was found. Sensors could now be placed on to the pads using just a small amount of Kapton tape or glue along the fingers (not on the exposed copper) without the need for soldering. The entire device would then be covered in two sheets of a transparent and sterile bandage, which would hold the sensors in place while keeping the device waterproof. It was also proposed that the device could be held in place in a subject's mouth using denture paste. The practicality of the paste was tested and functioned successfully with the device, as the subject could drink water with the device remaining attached in position.

The programming module system could also be designed such that the programmer is a separate device that is temporarily connected to the microcontroller through pins or contacts. This would reduce the electronic waste of a disposable programming module system.

In an earlier contemplated embodiment, the dysphagia/biomedical sensor prototype consisted of four FSRs that were connected through wires to communicate readings to a rigid PCB board, The goal for this part of the project was to create a flexible system that would fit in the mouth including the sensors.

Early in the project, we came up with a design through strain gauges. We began research of the use of strain gauges in the engineering field. In the previous prototype FSR (forces sensing resistors) were used wrapped in biomedical material however, this portion did not sit in the mouth comfortably. Nor did this sensor allow for easy swallowing.

In our updated approach we decided to attempt to use strain gauges. However, they were not effective in creating this system. Strain gauges had to be arranged in perpendicular to the force of the tongue. The minimal amount of room we had didn't allow for any strain gauges to be placed. Strain gauges had to be applied using a strong adhesive to a certain material. In the engineering field, strain gauges are applied on different materials, this consist of bridges, pipelines and metal appliances. They are used for stress analysis as well as transducing mechanical stress to electrical signals. We made a design prototype that would help give us a start on recording data but, later on throughout our research we've realized that strain gauges are difficult to apply on certain materials without the correct adhesives. In addition, we couldn't fathom a material safe to apply on the strain gauge and also be safe in a person's mouth, and this final point was paramount in this work, since contemplated sensor systems are biomedical devices and must be suitable for patient use.

In one contemplated embodiment, an arrangement of FSR (Force Sensitive Resistors) was found to be a good fit. This contemplated embodiment needed small enough resistors to be placed in the mouth. However, the resistors needed to be cut off. The substrate we used was a thin Kapton material. Kapton is also a good material because it is able to hold its integrity from heat.

The plan was to solder the leads to a few enameled wires that would be connected to pads on the flexible PCB. We also explored the use of carbon nanotubing material and its potential to become a contemplated sensor. The biomedical sensor system will go through a redesign with the goal to utilize carbon nanotubing as a thin resistive material. Carbon nanotubing has the conductive potential to transduce voltage.

Another contemplated embodiment utilizes a PCB with the routes/fingerprints already built into the PCB. Then, our next step was to apply droplets of carbon nanotube onto the fingerprints. A cylinder shape prototype is also contemplated in order to better keep the material in a consistent shape and structure.

In one contemplated embodiment, we kept the FSRs and combined them with our new flexible PCB with fingerprints/routes ready to be soldered on. These sensors are able to fit on the PCB and function to record measurements.

### REFERENCES

[1] Y. Takei Et Al., “Anterior And Posterior Tongue Activity Sensor Based On Triaxial Force Sensor,” 2013 IEEE 26^{Th} International Conference On Micro Electromechanical Systems (Mems), 2013, Pp. 1093-1096.
[2] C.-H. Lu, M. O. Shaikh, H.-Y. Liu And C. H. Chuang, “Piezoresistive Tactile Sensor With Tongue Pressure Measurement Capability For Dysphagia Assessment,” 2020 Ieee International Conference On Consumer Electronics-Taiwan (ICCE-Taiwan), 2020, Pp. 1-2.
[3] A. Alotaibi And S. Anwar, “Direct And Extended Piezoresistive And Piezoelectric Strain Fusion For A Wide Band Pvdf/Mwcnt-Based 3D Force Sensor,” In IEEE Access, Vol. 9, Pp.
[4] lizuka, M., Kobayashi, M., Hasegawa, Y. Et Al. A New Flexible Piezoelectric Pressure Sensor Array For The Noninvasive Detection Of Laryngeal Movement During Swallowing. J Physiol Sci 68, 837-846 (2018).
[5] Kong, X. Y., & Chen, Q. (2005). Quantum Mechanical Modeling of the Piezoresistive Effect in Carbon Nanotubes. Nano Letters, 5 (4), 667-672.
[6] Stampfer, C., Helbling, T., Obergfell, D., Schoberle, B., Tripp, M. K., Jungen, A., Roth, S., Bright, V. M., & Hierold, C. (2006). Fabrication of single-walled carbon nanotube-based pressure sensors. Nano Letters, 6 (2), 233-237.

Thus, specific embodiments, methods of flexible hybrid circuits, along with systems of embedded processing and wireless communications have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A biomedical sensor system, comprising:
a piezoresistive agent, and
a flexible substrate, wherein the flexible substrate and the piezoresistive agent are coupled together to form the biomedical sensor system, and wherein the sensor system is removably located in or on a ceiling of a patient's mouth.

2. The sensor system of claim 1, wherein the sensor system measures applied tongue pressure on the ceiling of the patient's mouth.

3. The sensor system of claim 1, further comprising at least one additional active component.

4. The sensor system of claim 3, wherein the at least one additional active component comprises at least one of the following alone or in combination: batteries, resistors, carbon nanotubes, material adhesives, bioadhesives, wireless communication technology and components, microcontrollers, embedded processors, and related components.

5. The biomedical sensor system of claim 1, wherein the piezoresistive agent comprises a piezoresistive sensor.

6. The biomedical sensor system of claim 5, wherein the piezoresistive sensor comprises at least one carbon nanotube.

7. The biomedical sensor system of claim 6, wherein the piezoresistive sensor is activated or triggered electronically by mechanical deformation of at least one of the at least one carbon nanotube.

8. The biomedical sensor system of claim 7, wherein the mechanical deformation is caused by pressure on the ceiling of a patient's mouth by a tongue of the patient.

9. The biomedical sensor system of claim 1, wherein the flexible substrate comprises a wireless embedded microcontroller system and a communications system.

10. The biomedical sensor system of claim 1, wherein the communications system comprises Bluetooth communications protocol.

11. A biomedical sensor system, comprising:
a piezoresistive agent,
a flexible substrate, and
a patient having a mouth, a tongue, and a mouth ceiling, wherein the flexible substrate and the piezoresistive agent are coupled together to form the biomedical sensor system, and wherein the sensor system is removably located in or on the ceiling of the patient's mouth.

12. The biomedical sensor system of claim 11, wherein the flexible substrate additionally comprises a flexible hybrid circuit structure.

13. The biomedical sensor system of claim 12, wherein the flexible hybrid circuit structure comprises at least one additional active component,

14. The biomedical sensor system of claim 13, wherein the at least one additional active component comprises at least one of the following alone or in combination: batteries, resistors, carbon nanotubes, material adhesives, bioadhesives, wireless communication technology and components, microcontrollers, embedded processors, and related components.

15. A method of determining a level of dysphagia in a patient, the method comprising:
providing a biomedical sensor system, wherein the system comprises:
a piezoresistive agent, and
a flexible substrate comprising a flexible hybrid circuit structure, wherein the flexible substrate and the piezoresistive agent are coupled together to form the biomedical sensor system;
providing a patient having a mouth, a tongue, and a mouth ceiling,
removably locating the sensor system in or on the ceiling of the patient's mouth.

16. The method of claim 15, wherein the flexible hybrid circuit structure comprises at least one additional active component,

17. The method of claim 16, wherein the at least one additional active component comprises at least one of the following alone or in combination: batteries, resistors, carbon nanotubes, material adhesives, bioadhesives, wireless communication technology and components, microcontrollers, embedded processors, and related components.
